(19) United States
(12) Patent Application Publication
(10) Pub. No.: US 2011/0236893 A1
(43) Pub. Date: Sep. 29, 2011

(54) LGR5 MODULATORS IN THE TREATMENT OF ALOPECIA

(75) Inventor: Sandrine Rethore, Valbonne (FR)

(73) Assignee: GALDERMA RESEARCH & DEVELOPMENT, Les Templiers (FR)

(21) Appl. No.: 13/120,104

(22) PCT Filed: Sep. 21, 2009
(86) PCT No.: PCT/FR2009/051771

§ 371 Date: Jun. 3, 2011

(30) Foreign Application Priority Data
Sep. 19, 2008 (FR) 0856316

(51) Int. Cl. C12Q 1/68; G01N 33/68; C07H 21/00

(52) U.S. Cl. 435/6.11; 435/6.13; 435/7.92; 436/501; 536/23.1

(57) ABSTRACT

An in vitro method of screening for candidate compounds for the preventive or curative treatment of alopecia, which comprises determining the ability of a compound to modulate the expression or the activity of the LGR5 receptor is described. The use of modulators of the expression or of the activity of this receptor for treating alopecia is also described. In addition, methods for the diagnosis or prognosis, in vitro, of this disease are described.

The invention relates to the identification and the use of compounds which are modulators of the LGR5 receptor, for the treatment of alopecia. It also relates to methods for the in vitro diagnosis or in vitro prognosis of this pathological condition.

In human beings, hair growth is cyclical and comprises three successive phases: the anagen phase, the catagen phase and the telogen phase. Each follicle of the head of hair is therefore continuously renewed, in a cyclical manner and independently of the adjacent follicles (Kligman 1959, Montagna and Parakkal, 1974). The anagen phase or growth phase, during which the hair extends, lasts several years. This phase recapitulates the morphogenesis of the hair and is divided into 7 different stages (anagen I to anagen VII) (Muller-Rover et al., 2001). To simplify, the anagen phase is generally reduced to three steps which each group together several stages: early for steps I-III, middle of anagen for steps IV to V and late anagen for steps VI and VII.

The catagen phase which follows on from the anagen phase is very short and lasts only a few weeks. This phase is divided into 8 different stages (catagen I to catagen VIII) (Muller-Rover et al., 2001). During this phase, the hair undergoes involution, the follicle atrophies and its dermal implantation appears increasingly high. The telogen phase, which lasts a few months, corresponds to a resting period for the follicle, where the hair ends up falling out. After this resting phase, a new follicle is regenerated, on site, and a new cycle recommences (Montagna and Parakkal, 1974).

At each moment, not all the hairs are in the same phase at the same time. Thus, out of the approximately 150 000 hairs which make up a head of hair, only approximately 10% of them are at rest and will therefore be replaced in a few months according to a biological clock specific to each hair (Montagna, 1974).

In mice and the other mammals with fur, the hair follicles also have a renewal cycle comprising the three anagen, catagen and telogen phases, divided up into various stages. On the other hand, the hair cycles of young animals are often “synchronized”, i.e. in the same phase of the cycle at the same moment in the same anatomical region (Muller-Rover et al., 2001).

Natural hair loss is a physiological phenomenon which occurs continuously and can be estimated, on average, at a few hundred hairs per day for a normal physiological state. However, it so happens that the hair cycle can become disturbed and that hair loss accelerates and results in a temporary or permanent hair loss called alopecia. Various causes may be responsible for alopecia.

Various types of alopecia exist, the main forms being:

hereditary androgenetic alopecia, which is the most common: it manifests itself through a decrease in hair volume, or even baldness, and effects 70% of men;

acute alopecia: it can be associated with chemotherapy treatment, stress, substantial dietary deficiencies, iron deficiency, hormonal disorders, AIDS, acute irradiation;

alopecia areata which appears to be of autoimmune origin (cell-mediated mechanism), which is characterized by more or less large patches of baldness in one or more areas. This form of alopecia can affect the entire head, in which case the term alopecia totalis is used, and sometimes the entire body, then being referred to as alopecia universalis, and in this case there is no longer any body hair or head hair on the entire body.

In all these three cases, the hair loss is directly related to the hair cycle, the follicle no longer entering into the anagen phase, or the anagen phase not being maintained, which implies that the follicle no longer produces a hair shaft and therefore no longer produces hair. In order to combat alopecia, it is therefore necessary to reinitiate the hair cycle by activating the anagen phase.

Compositions which make it possible to suppress or reduce alopecia, and in particular to induce or stimulate entry into the anagen phase or hair growth, have been sought for many years in the cosmetics or pharmaceutical industry.

The applicant has now found that the gene encoding LGR5 is expressed specifically in hair follicle keratinocytes, and that its expression is induced at the moment of entry into anagen, in vivo, in a model of anagen entry induction by gonadectomy. It consequently proposes targeting this gene or its expression product, for preventing or improving alopecia phenomena.

The term “alopecia” is intended to mean all the forms of alopecia, namely, in particular, androgenetic alopecia, acute alopecia or alopecia areata.

### LGR5

The LGR5 gene encodes an orphan G protein-coupled receptor. LGR5, also known as GPR49, HG38 or FEX, is a protein which has an extracellular portion constituted of 18 “LRR” (leucine-rich repeat) units and a transmembrane portion.

In the context of the invention, the term “LGR5 gene” or “LGR5 nucleic acid” signifies the gene or the nucleic acid sequence which encodes the LGR5 protein. While the gene targeted is preferably the human gene or its expression product, the invention can also make use of cells expressing the LGR5 receptor by genomic integration or transient expression of an exogenous nucleic acid encoding the receptor.

The human nucleic sequence (SEQ ID No. 1) and the human protein sequence (SEQ ID No. 2) of the LGR5 receptor are reproduced in the attached appendix.

LGR5 is known to play an important role in the Wnt signalling cascade.

The Wnt signalling cascade is a pathway involved in cell proliferation and determination. In the absence of Wnt signal, the β-catenin cytoplasmic protein associates with a destruction complex containing various proteins, axin, the GSK-3β kinase (glycogen synthase kinase-3β and APC (adeomatosis polyposis coli). By attaching to this complex, β-catenin is phosphorylated and ubiquitinated, which leads to its degradation. The Wnt pathway is involved in the development of the tegumentary appendages (feathers, hairs, glands) but also plays a role during the hair cycle. The specific expression of LGR5 in the keratinocytes of the hair and its induction during entry into anagen suggests that it plays an important role in hair follicle homeostasis.

### Diagnostic Applications

A subject of the invention concerns an in vitro method for the diagnosis or the monitoring of the development of alopecia in an individual, comprising the comparison of the expression or of the activity of the LGR5 protein, of the expression of its gene or of the activity of at least one of its promoters, in a biological sample from an individual, compared with a control individual.

The expression of the protein can be determined by assaying this LGR5 protein by means of an immunohistochemical test or immunoassay (is it the same thing?), for example by ELISA assay. Another method, in particular for measuring the expression of the gene, is to measure the amount of corresponding mRNA, by any method as described above. Assaying of the activity of the LGR5 receptor can also be envisioned.

In the context of a diagnosis, the “control” individual is a “healthy” individual.

In the context of monitoring of the development of alopecia, the “control individual” refers to the same individual at a different time, which preferably corresponds to the beginning of the treatment (T0). This measurement of the difference in expression or in activity of the LGR5 protein, in the expression of its gene or in the activity of at least one of its promoters makes it possible in particular to monitor the efficacy of a treatment, in particular a treatment with an LGR5 transmembrane receptor modulator, as envisioned above or with another treatment against alopecia. Such monitoring can reassure the patient with regard to the well-founded nature of this treatment or the need to continue this treatment.

Another aspect of the present invention concerns an in vitro method for the determination of the predisposition of an individual to developing alopecia, comprising the comparison of the expression or of the activity of the LGR5 protein, of the expression of its gene or of the activity of at least one of its promoters, in a biological sample from an individual, compared with a control individual.

Here again, the expression of the protein can be determined by assaying the LGR5 protein, by means of an immunohistochemical test or immunoassay, for example by ELISA assay. Another method, in particular for measuring the expression of the gene, is to measure the amount of corresponding mRNA by any method as described above. Assaying of the activity of the LGR5 receptor can also be envisioned.

The individual tested is in this case an asymptomatic individual, exhibiting no hair disorder linked to alopecia. The “control” individual, in this method, signifies a “healthy” reference population or individual. The detection of this predisposition makes it possible to set up a preventive treatment and/or increased monitoring of the signs linked to alopecia.

In these methods for in vitro diagnosis or prognosis, the biological sample tested can be any sample of biological fluid or a sample of a biopsy. The sample may preferably be, however, a preparation of skin cells, obtained for example by hair removal or biopsy.

### Screening Methods

Another subject of the invention is an in vitro method of screening for candidate compounds for the preventive and/or curative treatment of alopecia, comprising the determination of the ability of a compound to modulate the expression or the activity of the LGR5 receptor or the expression of its gene or the activity of at least one of its promoters, said modulation indicating the usefulness of the compound for the preventive or curative treatment of alopecia. The method therefore makes it possible to select the compounds capable of modulating the expression or the activity of the LGR5 receptor, or the expression of its gene, or the activity of at least one of its promoters.

More particularly, the invention relates to an in vitro method of screening for candidate compounds for the preventive and/or curative treatment of alopecia, comprising the following steps:

a. preparing at least two biological samples or reaction mixtures;

b. bringing one of the samples or reaction mixtures into contact with one or more of the test compounds;

c. measuring the expression or the activity of the LGR5 protein, the expression of its gene or the activity of at least one of its promoters, in the biological samples or reaction mixtures;

d. selecting the compounds for which a modulation of the expression or of the activity of the LGR5 protein, of the expression of its gene or of the activity of at least one of its promoters is measured in the sample or the mixture treated in b), compared with the nontreated sample or mixture.

The term “modulation” is intended to mean any effect on the level of expression or of activity of the LGR5 receptor, of the expression of its gene or of the activity of at least one of its promoters, namely optionally an inhibition, but preferably a stimulation, which is partial or complete.

Thus, the compounds tested in step d) above preferably induce the expression or the activity of the LGR5 protein, the expression of its gene or the activity of at least one of its promoters.

Throughout the present text, unless otherwise specified, the term “expression of a protein” is intended to mean the amount of this protein;

the term “activity of a protein” is intended to means its biological activity;

the term “activity of a promoter” is intended to mean the ability of this promoter to initiate the transcription of the DNA sequence encoded downstream of this promoter (and therefore indirectly the synthesis of the corresponding protein).

The compounds tested may be of any type. They may be of natural origin or may have been produced by chemical synthesis. This may involve a library of structurally defined chemical compounds, of uncharacterized compounds or substances, or of a mixture of compounds.

Various techniques can be used to test these compounds and to identify the compounds of therapeutic interest, which modulate the expression or the activity of the LGR5 transmembrane receptor.

According to a first embodiment, the biological samples are cells transfected with a reporter gene functionally linked to all or part of the promoter of the LGR5 gene, and step c) described above consists in measuring the expression of said reporter gene.

The reporter gene may in particular encode an enzyme which, in the presence of a given substrate, results in the formation of coloured products, such as CAT (chloramphenicol acetyltransferase), GAL (beta-galactosidase) or GUS (beta-glucuronidase). It may also be the luciferase or GFP (green fluorescent protein) gene. The assaying of the protein encoded by the reporter gene, or of its activity, is carried out conventionally, by colorimetric, fluorometric or chemiluminescence techniques, inter alia.

According to a second embodiment, the biological samples are cells expressing the gene encoding the LGR5 receptor, and step c) described above consists in measuring the expression of said gene.

The cell used in this case may be of any type. It may be a cell expressing the LGR5 gene endogenously, for instance a liver cell, a prostate cell, or better still a skin cell, hair follicle keratinocytes or dermal papilla fibroblasts. Organs of human or animal origin, for instance hair, or whisker hair follicles, may also be used.

It may also be a cell transformed with a heterologous nucleic acid encoding the LGR5 transmembrane receptor, said cell preferably being human or mammalian.

A wide variety of host cell systems can be used, for instance Cos-7, CHO, BHK, 3T3 or HEK293 cells. The nucleic acid can be stably or transiently transfected, by any method known to those skilled in the art, for example by means of calcium phosphate, DEAE-dextran, liposome, virus, electroporation or microinjection.

In these methods, the expression of the LGR5 gene can be determined by measuring the transcription rate of said gene or its translation rate.

The term “transcription rate of a gene” is intended to mean the amount of corresponding mRNA produced. The term “translation rate of a gene” is intended to mean the amount of corresponding protein produced.

Those skilled in the art are familiar with the techniques for the quantitative or semi-quantitative detection of the mRNA of a gene of interest. Techniques based on hybridization of mRNA with specific nucleotide probes are the most common (Northern blotting, RT-PCR, Rnase protection). It may be advantageous to use detection labels, such as fluorescent, radioactive or enzymatic agents or other ligands (for example, avidin/biotin).

In particular, the expression of the gene can be measured by real-time PCR or by RNase protection. The term “RNase protection” is intended to mean the detection of a known mRNA among the poly(A)-RNAs of a tissue, which can be carried out by means of specific hybridization with a labelled probe. The probe is a labelled complementary RNA (for example radioactively or enzymatically labelled) of the messenger to be sought. It can be constructed from a known mRNA of which the cDNA, after RT-PCR, has been cloned into a phage. Poly(A)-RNA of the tissue in which the sequence is to be sought is incubated with this probe under slow hybridization conditions in a liquid medium. RNA:RNA hybrids form between the mRNA being sought and the antisense probe. The medium hybridized is then incubated with a mixture of ribonucleases specific for single-stranded RNA, such that only the hybrids formed with the probe can withstand this digestion. The digestion product is then deproteinized and repurified, before being analysed by electrophoresis. The labelled hybrid RNAs are detected, for example, by autoradiography or chemiluminescence.

The rate of translation of the gene is evaluated, for example, by immunoassay of the product of said gene. The antibodies used for this purpose may be of polyclonal or monoclonal type. The production of said antibodies falls within the context of conventional techniques. An anti-LGR5 polyclonal antibody can, inter alia, be obtained by immunization of an animal, such as a rabbit or a mouse, with the whole protein. The antiserum is collected and then depleted according to methods known per se by those skilled in the art. A monoclonal antibody can, inter alia, be obtained by the conventional method of Köhler and Milstein (Nature (London), 256: 495-497 (1975)). Other methods for preparing monoclonal antibodies are also known. It is possible, for example, to produce monoclonal antibodies by expression of a clone nucleic acid from a hybridoma. It is also possible to produce antibodies by the phage display technique, by introducing antibody cDNAs into vectors, which are typically filamentous phages that display V-gene libraries at the surface of the phage (for example, fUSE5 for E. coli).

The immunoassaying can be carried out in solid phase or in homogeneous phase; in one step or in two steps; in a sandwich method or in a competition method, by way of nonlimiting examples. According to one preferred embodiment, the capture antibody is immobilized on a solid phase. By way of nonlimiting examples of a solid phase, use may be made of microplates, in particular polystyrene microplates, or solid particles or beads, or paramagnetic beads.

ELISA assays, immunoassays or any other detection technique can be used in order to reveal the presence of the antigen-antibody complexes formed.

The characterization of the antigen/antibody complexes, and more generally of the isolated or purified but also recombinant proteins (obtained in vitro and in vivo), can be carried out by mass spectrometry analysis. This identification is made possible through the analysis (determination of the mass) of the peptides generated by enzymatic hydrolysis of the proteins (in general trypsin). In general, the proteins are isolated according to the methods known to those skilled in the art, prior to the enzymatic digestion. The analysis of the peptides (in hydrolysate form) is carried out by separation of the peptides by HPLC (nano-HPLC) based on their physicochemical properties (reverse phase). The determination of the mass of the peptides thus separated is carried out by peptide ionization and either by direct coupling with mass spectrometry (ESI electrospray mode) or after deposition and crystallization in the presence of a matrix known to those skilled in the art (analysis in MALDI mode). The proteins are then identified through the use of appropriate software (for example Mascot).

The LGR5 receptor can be produced according to customary techniques using Cos-7, CHO, BHK, 3T3 and HEK293 cells. It can also be produced by means of microorganisms such as bacteria (for example, E. coli or B. subtilis), yeasts (for example Saccharomyces, Pichia) or insect cells, such as Sf9 or Sf21.

### LGR5 Receptor Modulators

A subject of the invention is also the use of an LGR5 receptor modulator which can be obtained according to one of the methods described above, for the preparation of a medicament for use in the preventive and/or curative treatment of alopecia.

A method for the preventive and/or curative treatment of alopecia, said method comprising the administration of a therapeutically effective amount of an LGR5 receptor modulator, to a patient requiring such a treatment, is thus described herein.

Preferably, such modulators are LGR5 receptor activators (or inducers).

The invention comprises the use of compounds which are LGR5 receptor inducers, such as those identified by the screening method described above, for the preventive and/or curative treatment of alopecia.

The modulator compounds are formulated in pharmaceutical compositions, in combination with a pharmaceutically acceptable vehicle. These compositions can be administered, for example, enterally, parenterally or topically. Preferably, the pharmaceutical composition is applied topically. Via oral administration, the pharmaceutical composition can be in the form of tablets, gelatin capsules, sugar-coated tablets, syrups, suspensions, solutions, powders, granules, emulsions, suspensions of microspheres or nanospheres or lipid or polymeric vesicles for controlled release. Via parenteral administration, the pharmaceutical composition can be in the form of solutions or suspensions for infusion or for injection.

By topical application, the pharmaceutical composition is more particularly for use in treating the skin, the mucous membranes or the scalp and can be in the form of salves, creams, milks, ointments, powders, impregnated pads, solutions, gels, sprays, lotions or suspensions. It may also be in the form of suspensions of microspheres or nanospheres or of lipid or polymeric vesicles or of polymeric patches or of hydrogels for controlled release. This composition for topical application may be in anhydrous form, in aqueous form or in the form of an emulsion. In one preferred variant, the pharmaceutical composition is in the form of a gel, a cream or a lotion.

The composition may comprise a content of LGR5 receptor modulator ranging from 0.001% to 10% by weight, in particular from 0.01% to 5% by weight, relative to the total weight of the composition.

The pharmaceutical composition may also contain inert additives or combinations of these additives, such as:
wetting agents;
taste enhancers;
preservatives such as para-hydroxybenzoic acid esters;
stabilizers;
water-content regulators;
pH regulators;
osmotic pressure modifiers;
emulsifiers;
UV-A and UV-B screening agents;
and antioxidants, such as alpha-tocopherol, butylhydroxyanisole or butylhydroxytoluene, superoxide dismutase, ubiquinol or certain metal-chelating agents.

The following figures and examples illustrate the invention without limiting the scope thereof.

### FIGURE LEGEND

FIG. 1 illustrates the induction of the transition into anagen by ovariectomy. Female mice, of which the hair follicles of the dorsal region were in telogen at day 0, were subjected or not subjected (control) to an ovariotomy on day 1 of the study. A sample of the skin from the region on the back of the mice was taken on days 0 and 8 of the study. FIG. 1A represents a histological section of skin from the dorsal region of a mouse on day 0 of the study. FIG. 1B represents a histological section of skin from the dorsal region of an ovariectomized mouse on day 8 of the study. FIG. 1C represents a histological section of skin from the dorsal region of a control mouse on day 8 of the study. The histological analysis clearly shows that the ovariectomy induced transition into anagen (FIG. 1B).

FIG. 2 is a table which gives the modulation of the level of expression of the LGR5/GPR49 receptor, expressed relative to day 0 of the study, in the skin of the dorsal region of ovariectomized mice on day 8 of the study and in the skin of the dorsal region of control mice (skin in telogen phase) on day 8 of the study, using the Affymetrix array technology. Female mice, of which the hair follicles of the dorsal region were in telogen at day 0, were subjected to an ovariotomy on day 1 of the study. Non-ovariectomized mice were retained so as to serve as a control group. A sample of the skin from the dorsal region of the mice was taken on days 0 and 8 of the study. The RNAs were isolated and the gene expression was analysed using the Affymetrix array technology.

FIG. 3 shows the expression of the LGR5/GPR49 receptor in mouse skin in telogen and at the beginning of anagen by in situ hybridization. FIG. 3A is the photograph of the black-background image of a section of mouse skin in telogen subjected to in situ hybridization using an antisense probe for the LGR5/GPR49 receptor; the histological structures radioactively labelled by the probe are revealed by the accumulation of luminous spots (silvery grains). FIG. 3B is the photograph of the same histological section of mouse skin in early anagen, counterstained with hematoxylin.

FIG. 3C is the photograph of the black-background image of a section of mouse skin in early anagen (III) subjected to in situ hybridization using an antisense probe for the LGR5/GPR49 receptor; the histological structures radioactively labelled with the probe are revealed by the accumulation of luminous spots (silvery grains). FIG. 3D is the photograph of the same histological section of mouse skin in late anagen, counterstained with hematoxylin.

FIG. 4 shows the expression of the LGR5/GPR49 receptor in mouse skin in late anagen and catagen by in situ hybridization. FIG. 4A is the photograph of the black-background image of a section of mouse skin in late anagen subjected to in situ hybridization using an antisense probe for the LGR5/GPR49 receptor; the histological structures radioactively labelled by the probe are revealed by the accumulation of luminous spots (silvery grains). FIG. 4B is the photograph of the same histological section of mouse skin in early anagen, counterstained with hematoxylin.

FIG. 4C is the photograph of the black-background image of a section of mouse skin in catagen subjected to in situ hybridization using an antisense probe for the LGR5/GPR49 receptor; the histological structures radioactively labelled by the probe are revealed by the accumulation of luminous spots (silvery grains). FIG. 3D is the photograph of the same histological section of mouse skin in late anagen, counterstained with hematoxylin.

### EXAMPLES

### Experimental Data

### Example I

Expression of the LGR5/GPR49 Receptor During Ovariectomy-Induced Entry into Anagen using the Affymetrix Array Technology

### Methods:

42-day-old female C57BL/6 mice of which the hair follicles of the dorsal region were in telogen (Chase, 1954) were optionally ovariectomized on day 1 of the study. Ovariectomy carried out during the telogen phase causes, within a week, a massive entry of the hair follicles of the dorsal region into the anagen phase (Chanda, 2000), whereas the hair follicles of the dorsal region of the control animals are still in telogen.

Skin samples were taken from the dorsal region on days 0, 6 and 8 of the study. One part of the sample was used to confirm the transition into anagen by histological analysis. The other part of the sample was used to carry out a transcriptome analysis using the Affymetrix array technology.

Gene expression was analysed on an Affymetrix station (microfluidic module; hybridization oven; scanner; computer) according to the supplier's recommendations. In summary, the total RNAs isolated from the tissues are transcribed into cDNA. The biotin-labelled cRNAs are synthesized, from double-stranded cDNA, using T7 polymerase and a biotin-conjugated NTP precursor. The cRNAs are then fragmented into fragments of small sizes. All the molecular biology steps are verified using the Agilent “Lab on a chip” system in order to confirm good efficiency of the enzymatic reactions. The Affymetrix array is hybridized with the biotinylated cRNA, rinsed and then labelled with fluorescence using a streptavidin-conjugated fluorophore. After various washes, the array is scanned and the results are calculated using the MAS5 software provided by Affymetrix. An expression value is obtained for each gene, along with the indication of the presence or absence of the value obtained. The calculation of the significance of the expression is based on the analysis of the signals which are obtained following the hybridization of the cRNA of a given gene with a perfect match oligonucleotide compared with a oligonucleotide which contains a mutation (single mismatch) in the central region of the oligonucleotide.

### Results:

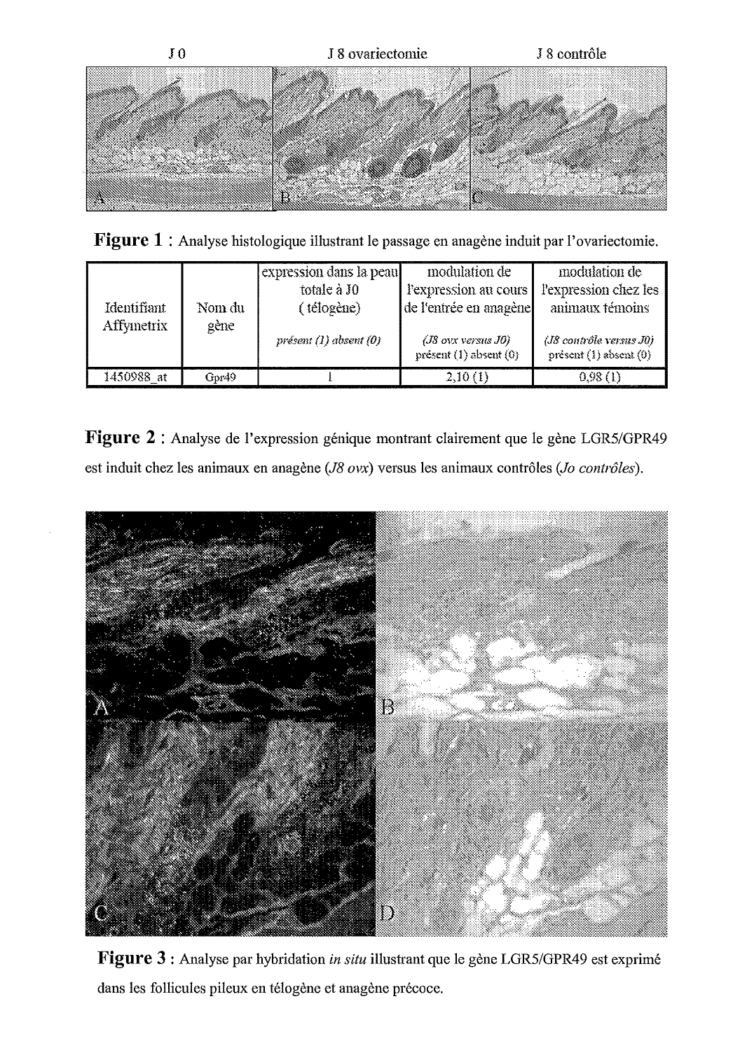

### FIG. 1:

At the beginning of the study on day 0, the histological analysis shows that the hair follicles of the dorsal region of the skin of the mice are in the telogen phase (1A). In the mice subjected to an ovariectomy, the hair follicles of the dorsal skin region are at the beginning of the anagen phase (1B). Conversely, the hair follicles of the dorsal region of skin of the control mice (non-ovariectomized) have remained in the telogen phase. Thus, the ovariectomy induced transition from the telogen phase to the anagen phase. The anagen phase is established by histological analysis on day 8 of the study.

### FIG. 2:

The LGR5/GPR49 receptor is expressed in the telogen phase and in the anagen phase of the hair cycle. The differential analysis between the expression at the telogen stage (at D0) and the anagen stage (D8 ovariectomized) shows that the expression of the LGR5/GPR49 receptor transcripts is induced in early anagen compared with the telogen stage, whereas, in the control mice, the expression of the LGR5/GPR49 receptor is not induced compared with the beginning of the study.

### Example 2

Expression of the LGR5/GPR49 Receptor in Mouse Skin using “in Situ Hybridization”

### Methods:

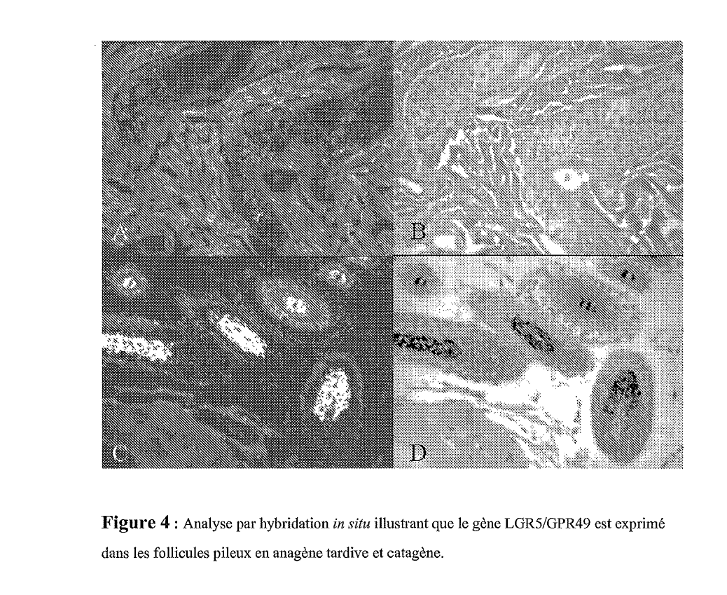

Sense and antisense probes were prepared from the Sox4 transcription factor by incubating the linearized gene (2 μg) with 63 μCi of [³⁵S] UTP (1250 Ci/mmol; NEN, Massachusetts, USA) in the presence of the T7 or T3 RNA polymerase. The in situ hybridization was carried out on a mouse tissue fixed with formaldehyde and embedded in paraffin. Sections (4 μm thick) were then deparaffinised in toluene and rehydrated in an alcohol gradient. After drying, the various sections were incubated in a prehybridization buffer for two hours. The hybridization was carried out overnight in a hybridization buffer (prehybridization buffer with 10 mM DTT and 2 10⁶ cpm RNA/μl, ³⁵S-labelled) at 53° C. The excess probe was removed and the sections were incline in an LM1 photographic emulsion (Amersham Biosciences, UK) and exposed in the dark at 4° C. for at least one month. The sections were then developed and counterstained with hematoxylin and eosin. Following the incubation in the presence of a photographic emulsion, the histological structures radioactively labelled with the probe are revealed (accumulation of silvery grains). A specific signal manifests itself through positive labelling with the antisense probe (FIG. 4B and FIG. 5B) and the absence of labelling with the sense probe (FIG. 3A and FIG. 4A), used as a negative control.

### Results:

### FIG. 3

The images (A to B) show hair follicles of skin from the back of mice in telogen. The images (C to D) show hair follicles of skin from the back of mice at the beginning of anagen (stage III). FIG. 3A shows that the LGR5/GPR49 receptor is expressed specifically in the hair follicles in mouse skin in telogen. More particularly, the LGR5/GPR49 receptor is present in the keratinocytes in contact with the dermal papilla. FIG. 3C shows that the LGR5/GPR49 receptor is expressed specifically in the hair follicles at the beginning of anagen in the keratinocytes which will form the new hair follicle.

### FIG. 4

The images (A to B) show hair follicles of skin from the back of mice in late anagen. The images (C to D) show hair follicles of skin from the back of mice in catagen. FIG. 3A shows that the LGR5/GPR49 receptor is expressed specifically in the external epithelial sheath of the hair follicles in mouse skin in late anagen. FIG. 3C shows that the LGR5/GPR49 receptor is expressed specifically in the hair follicles at the beginning of catagen.

### CONCLUSION

Example 1 shows that the LGR5/GPR49 receptor is expressed in the skin and induced during the entry into anagen. Example 2 emphasizes that the LGR5 gene is expressed specifically in the hair follicle keratinocytes at various stages of the hair cycle.

These studies as a whole make it possible to support the use of modulators of LGR5/GPR49 receptor expression in humans for obtaining a stimulation of hair follicle growth by inducing entry into the anagen phase. In addition, they support the advantage of using the LGR5/GPR49 receptor, for the diagnosis or prognosis of this pathological condition.

1. An in vitro method of screening for candidate compounds for treatment of alopecia, the method comprising determining the ability of a compound to modulate the expression or the activity of LGR5 or the expression of its gene or the activity of at least one of its promoters.

2. The method according to claim 1, further comprising the following steps:
a. preparing at least two biological samples or reaction mixtures;
b. bringing one of the samples or reaction mixtures into contact with one or more test compounds;
c. measuring the expression or the activity of the LGR5 protein, the expression of its gene or the activity of at least one of its promoters, in the biological samples or reaction mixtures; and
d. selecting the compounds for which a modulation of the expression or of the activity of the LGR5 protein, or a modulation of the expression of its gene or a modulation of the activity of at least one of its promoters is measured in the sample or the mixture treated in b), compared with the nontreated sample or mixture.

3. The method according to claim 2, wherein the compounds selected in step d) activate the expression or the activity of the LGR5 protein or the expression of its gene or the activity of at least one of its promoters.

4. The method according to claim 2, wherein the biological samples are cells transfected with a reporter gene functionally linked to all or part of the promoter of the gene encoding the LGR5 receptor, and in that step c) comprises measuring the expression of the reporter gene.

5. The method according to claim 2, wherein the biological samples are cells expressing the gene encoding the LGR5 receptor, and in that step c) comprises measuring the expression of the gene.

6. The method according to claim 4, wherein the cells are selected from a group consisting of keratinocytes and fibroblasts of the dermal papilla or of the dermis.

7. The method according to claim 4, in wherein the cells are cells transformed with a heterologous nucleic acid encoding the LGR5 receptor.

8. The method according to claim 2, in which the expression of the gene is determined by measuring the transcription rate of the gene.

9. The method according to claim 2, wherein the expression of the gene is determined by measuring the translation rate of the gene.

10. A medicament for the treatment of alopecia, the medicament comprising an effective amount of a LGR5 receptor modulator obtained according to claim 1.

11. The medicament according to claim 10, wherein the modulator is an activator of the LGR5 receptor.

12. A cosmetic for aesthetic treatment of the scalp, the cosmetic comprising an effective amount of a LGR5 receptor modulator.

13. An in vitro method for the diagnosis or the monitoring of the development of alopecia in an individual, the method comprising comparing the expression or of-the activity of the LGR5 protein, or the expression of its gene or the activity of at least one of its promoters, in a biological sample from an individual, with a biological sample from a control individual.

14. The method according to claim 13, wherein the expression of the protein is determined by assaying the protein with an immunoassay.

15. The method according to claim 14, wherein the immunoassay is an ELISA assay.

16. The method according to claim 13, which wherein the expression of the gene is determined by measuring the amount of corresponding mRNA.

17. An in vitro method for the determination of the predisposition of an individual to developing alopecia, the method comprising comparing the expression or the activity of the LGR5 protein, or the expression of its gene or the activity of at least one of its promoters, in a biological sample from an individual, compared with a biological sample from a control individual.
